(19) United States
(12) Patent Application Publication
(10) Pub. No.: US 2007/0275061 A1
(43) Pub. Date: Nov. 29, 2007

(54) PHARMACEUTICAL COMPOSITIONS AND FORMULATIONS OF METFORMIN EXTENDED RELEASE TABLETS

(76) Inventors: Young Gwan Jo, Daejeon (KR); Ja-Seong Koo, Daejeon (KR); Ju-Bin Yim, Daejeon (KR); Yoon-Sik Jun, Seoul (KR)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/534,359

(22) Filed: Sep. 22, 2006

(30) Foreign Application Priority Data
May 23, 2006 (KR) 10-2006-0046145

(51) Int. Cl. A61K 9/22; A61K 31/155

(52) U.S. Cl. 424/468; 514/635

(57) ABSTRACT

The present invention relates to a metformin extended release tablet, particularly to a metformin extended release tablet comprising metformin, which is effective in treating non-insulin dependent diabetes mellitus, as active ingredient, and a matrix in which hydrophilic polymers and hydrophobic materials are mixed at a specific proportion. The hydrophilic polymers enable controlling of the pore size of the gel layer formed by water swelling, thereby enabling primary control of drug release rate. And, the hydrophobic materials block the pores of the gel layer, thereby enabling secondary control of drug release rate. Therefore, the metformin extended release tablet of the present invention has better dissolution properties than conventional extended release tablets and, thus, enables extended drug release at constant rate even with less matrix. A constant blood level can be maintained with one administration a day and the tablet can be made smaller, which makes the administration easier and the production simpler.

### TECHNICAL FIELD

The present invention relates to a metformin extended release tablet, particularly to a metformin extended release tablet comprising metformin, which is effective in treating non-insulin dependent diabetes mellitus, as an active ingredient and a matrix in which hydrophilic polymers and hydrophobic materials are mixed at a specific proportion. The aforementioned hydrophilic polymers enable controlling of the pore size of the gel layer formed by water swelling, thereby enabling the primary control of drug release rate, while the aforementioned hydrophobic materials block the pores of the gel layer, thereby enabling the secondary control of drug release rate. Therefore, the metformin extended release tablet of the present invention has better dissolution properties than conventional extended release tablets and, thus, enables extended drug release at constant rate even with less matrix. A constant blood level can be maintained with one administration a day and the tablet can be made smaller, which makes the administration easier and the production procedure simpler, hence results in commercial profit.

### BACKGROUND ART

Metformin is a biguanide medication effective in treating non-insulin dependent diabetes mellitus. When administered to a diabetic, it controls blood sugar level by preventing glucose absorption in the gastrointestinal tract. Highly soluble in water, metformin may cause excessive reduction in blood sugar level when medicated in the form of an ordinary tablet.

Normally, the maximum administration dosage of metformin is 2550 mg per day. It is administered in the form of 500 mg- or 750 mg-tablet twice or three times a day with a meal (after a meal). But, such an administration method may cause abrupt change of the concentration of the drug in blood and, possibly, cause adverse reactions or resistance to the drug. Thus, an extended release tablet, which is designed to release the drug at a constant rate for 24 hours, is thought as the most preferable administration form in view of therapeutic effect as well as patient's convenience.

Since metformin hydrochloride is very highly soluble in water and is hardly permeable in the lower gastrointestinal tract, most of the drug has to be absorbed in the upper gastrointestinal tract, which makes the development of extended release tablets complicated. A lot of patents have been issued on metformin extended release tablets worldwide. However, they do not take into consideration of the physical properties or the narrow absorption window of metformin, or have a weakness of requiring high facility cost due to complex and complicated preparation processes.

Drugs with narrow absorption window like metformin hydrochloride require extension of residence time by swelling in the gastrointestinal tract. Also, the administration form has to be an extended release form adequate for production in commercial scale.

However, such commonly known extended release administration forms as the osmotic release type using semipermeable coating, the controlled release type using enteric coating or the controlled release type utilizing control of elution rate of granules, are not adequate for metformin, considering the narrow absorption window of the drug. Moreover, they require expensive equipments for production.

Andrex disclosed a method of treating a pharmaceutical composition with a semipermeable coating then forming holes with a laser drill [PCT/US 1999/06024]. The aforementioned method is disadvantageous as the laser drill itself is an expensive tool while the size of the holes through which the drug is released may be non-uniform, depending on who performs the drilling or under what situation the drilling is being performed. Since the drug release profile shows a great deviation depending upon the hole size, this method is not adequate for producing the treatments for the diabetes, while the production cost is also high.

In U.S. Patent Application No. 2004/0161461, Sethpawan disclosed a method of dissolving a binder in a solvent, adding a swelling agent to form granules, drying and making the granules into a tablet and forming a semipermeable coating on the tablet. The aforementioned method may not be considered superior production method, as the coating process requires an elaborate manipulation and it is not easy to attain a uniform coating.

Sanghri and Pradeep proposed applying a natural gum, such as xanthan gum and locust bean gum, to metformin salt in U.S. Patent Application No. 2004/0109891. However, in this case, calcium sulfate or plaster used as an ionization agent is insoluble in water and, thus, may not properly function as an ionization agent and form gel with the natural material.

In U.S. Pat. No. 6,682,759, Jong C. Lim and John N. Shell proposed two-phase controlled release in which an immediate-release layer is coated over a prolonged-release core.

In the aforementioned patent, it is not very difficult to obtain the prolonged-release layer with technically uniform quality, yet it is very difficult to obtain the immediate-release layer with uniform thickness, as it has to be prepared by wet coating. Moreover, this method cannot be considered a superior technology as it has a stability problem such as reduced titer while it is difficult to ensure uniform quantity of immediate-release drugs.

Kumar Gidwani et al. proposed in U.S. Patent Application No. 2004/0076667 a method of melting fatty acid and fatty acid ester at high temperature, making them to granules and preparing them into a tablet. However, this process, also, is very complicated and the drug may be decomposed at high temperature.

Zhang and Xiaoying proposed in U.S. Patent Application No. 2004/0086566, a method of mixing metformin with wax and preparing a tablet by hot melting, in the similar manner proposed by Kumar Gidwani above.

And, in U.S. Pat. No. 6,676,966, Amina Odidi and Isa Odidi disclosed an extended release tablet using a methacrylic acid copolymer as coating material, in which the coat layer is dissolved depending on the pH. The coat layer is stable under acidic pH but is dissolved at pH 5-6 or higher. In other words, since the tablet is not dissolved under acidic pH but is dissolved under weakly acidic pH (pH 5-6), metformin is not absorbed in the upper gastrointestinal tract.

Considering the large unit quantity for the administration of metformin and the chemicophysical properties of high solubility and poor compatibility, along with the narrow absorption window, the extended release systems proposed by the preceding patents are imperfect formulations for metformin.

Needless to say, the floating type extended release dosage form using polyvinylpyrrolidone (U.S. Pat. No. 6,635,279) can be said to be adequate for narrow absorption window. However, bioavailability may appear different depending on the condition of foods inside the gastral cavity. Thus, in case the unit quantity is large and highly soluble metformin is used as the main component, the tablet may be disintegrated quickly and cannot ensure consistent extended release over 24 hours. In addition, metformin is not easy to be made into a tablet due to its poor compacting and compounding properties. Therefore, the most suitable extended release formulation for metformin appears to be the water swelling type controlled release system using hydrophilic polymers.

The conventional preparation forms of metformin are inadequate when considering the narrow absorption window, solubility, compactability and unit dose of metformin. Considering the narrow absorption window of the upper gastrointestinal tract and the possibility of using existing facilities, the water swelling type controlled release system using hydrophilic polymers is the best candidate. However, for such preparation forms that require a large unit dose of 500 mg to 750 mg as metformin, the total weight increases greatly and the resulting tablet may be too large-sized to be administered orally.

Although the controlled release of Depomed (International Application No. PCT/US 1998/55107) is a water swelling type controlled release system, it is less effective than the present invention of sustaining controlled release over 24 hours, in that all the active ingredients are released within 8 hours. In addition, it does not specify particular material composition or design that enables the extended release. For such drugs that require a large unit dose while having a poor compactability as metformin, it is impossible to attain a controlled release over 24 hours using general polymers only. And, the resultant tablet becomes too large-sized to be administered orally. In other words, the controlled release over 8 hours attained by the patent can be said to be the limit for the extended release of the systems using general polymers.

In other words, metformin requires a relatively large unit dose and, thus, the size for the preparation in the form of tablet or capsule has to be massive. Also, since it has a very high solubility, a relatively large amount of polymers has to be used, which makes the preparation form even bigger.

The large unit dose and the high solubility may cause disintegration of the matrix and, thereby, may interrupt the controlled release. If a large amount of matrix is used to attain the desired controlled release, the preparation form becomes too large-sized to be taken-in. And, if the amount of matrix is decreased to make a moderate-sized preparation form, the effect of controlled release is reduced. Therefore, designing of a preparation form that enables consistent controlled release over 24 hours with a small amount of matrix is an important task in attaining metformin extended release tablets of practical use.

Accordingly, it is very important to develop a controlled release system enabling control of drug release with a small amount of polymers.

### DISCLOSURE OF THE INVENTION

The inventors of this invention worked to solve these problems and developed an extended release tablet capable of effectively controlling release of metformin, which requires a large unit dose and has high solubility.

As a result, they completed this invention with a finding that the drug release can be controlled with a relatively small amount of matrix through a systematic controlled release by mixing hydrophilic polymers with hydrophobic materials.

In this invention, a matrix comprising at least two hydrophilic polymers and at least one hydrophobic material is used to control the released speed, hence enabling consistent drug release over 24 hours.

In other words, this invention provides a water swelling type controlled release system, in which the hydrophilic polymers increases the viscosity of the gel layer formed by water swelling as the matrix contacts the released drug and reduces the pore size of the gel layer, thereby enabling the primary control of drug release rate, and the hydrophobic materials block the pores, thereby enabling the secondary control of drug release rate. To put it otherwise, because release of the drug caused by the disintegration of the gel layer determines the release rate, the use of two or more hydrophilic polymers improves viscosity and cohesiveness of the gel layer, thereby enabling extended release of the drug component. And, the hydrophobic materials interrupt the release of the drug through the pores, thereby further improving the control of the extended release.

Furthermore, since the residence time of the drug in the gastrointestinal tract can be increased by the use of metformin, the active drug component, and the matrix for controlling the release rate of metformin, the rate of absorption of metformin in the gastrointestinal tract can be controlled. In accordance with this invention, the drug release is sufficiently sustained over 24 hours with smaller amount of matrix than is used for the preparation of conventional extended release tablets. Thus, the size of the tablet can be reduced for more convenient medication.

Accordingly, the aim of this invention is to provide a metformin extended release tablet enabling sustained release of metformin in the body, capable of being simply prepared into a commercial scale and enabling consistent, controlled release of the drug even with a small amount of matrix, thereby making medication convenient with reduced size and being effective in reducing the production cost.

### DETAILED DESCRIPTION OF INVENTION

The current invention is characterized by a metformin extended release tablet comprising metformin as an active ingredient and a pharmaceutically acceptable mixture of hydrophilic polymers and hydrophobic materials as matrix.

Hereunder is given a more detailed description of the invention.

This invention is related to an improved metformin extended release tablet comprising metformin, which is the active ingredient effective in treating non-insulin dependent diabetes mellitus, and a matrix, prepared by mixing hydrophilic polymers and hydrophobic materials in specific proportion. The aforementioned hydrophilic polymers enable controlling of the pore size of the gel layer formed by water swelling, thereby enabling the primary control of drug release rate, while the hydrophobic materials block the pores of the gel layer, thereby enabling the secondary control of drug release rate. Therefore, the metformin extended release tablet of the present invention has better dissolution properties than the conventional extended release tablets and, thus, enables extended drug release at constant rate even with less matrix. A constant blood level can be maintained with one administration a day and the tablet can be made smaller, which makes the medication easier and the production simpler.

Hereunder is given a more detailed description of the metformin extended release tablet of this invention.

The metformin extended release tablet of this invention comprises metformin as active ingredient and a pharmaceutically acceptable mixture of hydrophilic polymers and hydrophobic materials as extended release matrix.

The metformin may be in the form of a pharmaceutically acceptable salt, including acid adduct, yet, metformin hydrochloride is most preferred. The following description is mainly on metformin hydrochloride, but the scope of the current invention is not restricted to the salt of metformin hydrochloride.

The metformin may be used in the range of 25-75 wt % of the total weight of the extended release tablet, preferably in the range of 30-70 wt %, and most preferably in the range of 35-65 wt %.

The metformin extended release tablet of this invention also comprises a matrix comprising a mixture of pharmaceutically acceptable hydrophilic polymers and hydrophobic materials.

When the matrix comprising a water swelling type hydrophilic polymer gets in contact with the released drug, gelation begins with the formation of water swelling. Structurally, the matrix can be divided into a portion where it contacts with the released drug and the portion where the released drug slowly penetrates and causes water swelling of the matrix. The portion where the matrix gets in contact with the released drug is the interface between the gel layer and the released drug, while the portion where the water swelling occurs is the interface between the gel layer and the not-yet-hydrated polymer.

The active drug ingredient is consecutively distributed in the swollen polymer matrix, and as the water swelling type hydrophilic polymer in the matrix is gelated by water swelling, the drug ingredient is released restrictively as being included in the gel.

The release profile of the drug ingredient is determined by the thickness of the hydrated gel layer, pore size of the gel layer and other gel situations. The thickness of the aforementioned gel layer is determined by the rate of penetration of water at the interface of the gel layer and the not-yet-hydrated polymer and the rate of erosion of the gelated matrix by water at the interface between the gel layer and the released drug. Meanwhile, the pore size of the gel layer is determined by the properties of the hydrophilic polymers comprising the matrix.

Drug components which are highly soluble in water like metformin are dissolved, as the matrix is gelated and are diffused and released out through the pores of the gel layer. Therefore, the rate of drug release is determined by many factors, including water swelling rate of the gel layer, the rate of erosion of the gel layer at the interface between the gel layer and the released drug, the pore size of the gel layer, and the viscosity and the properties of the gel, etc.

In other words, to attain consistent and uniform controlled release of highly soluble drug components, like metformin, it is required to reduce the pore size of the gel layer in order to control the drug release by diffusion or to reduce the water swelling rate of the gel layer in order to control the gelation of the matrix and decrease the drug release rate at the hydrated gel layer. Also, it is required to increase the viscosity of the gel layer or to make the gel harder, in order to reduce the water swelling rate and to reduce the rate of drug release by erosion or to make the matrix layer thicker in order to reduce the rate of the drug release through the matrix by diffusion.

Nevertheless, if the matrix layer is too thick, the tablet becomes too large-sized. Therefore, in case of drugs which require a large unit dose like metformin, it is advisable to control other release rate determining factors rather than controlling the rate of the drug release by making the matrix layer thicker.

That is, it is more appropriate to control the pore size of the gel layer, reduce the water swelling rate, or control the viscosity or the solidity of the gel layer to control the release rate.

In consideration of the factors determining the release rate described above, the current invention provides a metformin extended release tablet, capable of controlling the release profile and the release rate of such drug component as metformin over 24 hours, using less polymers as matrix than in the conventional methods.

For the hydrophilic polymers, a mixture comprising two or more molecules from saccharides, cellulose derivatives, gums, proteins, polyvinyl derivatives, polymethacrylate copolymers, polyethylene derivatives and carboxyvinyl polymers is used.

It is somewhat restricted to reduce the release rate of the drug component using a single water swelling type hydrophilic polymer.

Thus, a mixture of at least two water swelling type hydrophilic polymers is used in this invention. In other words, a gel layer with a higher viscosity is formed with less water swelling type hydrophilic polymers utilizing the crosslinkage of different water swelling type hydrophilic polymers, thereby reducing the pore size. In addition, the use of at least two water swelling type hydrophilic polymers results in the formation of pores in the gel layer with different sizes, thereby retarding the diffusion of the drug component through the pores of the gel layer and reducing the rate of drug release at the interface of the gel layer and the released drug, by an erosion with higher viscosity of the gel layer.

As for the saccharides, dextrin, polydextrin, dextran, pectin, pectin derivatives, alginate, polygalacturonic acid, xylan, arabinoxylan, arabinogalactan, starch, hydroxypropyl starch, amylose, amylopectin, etc. may be used. As for the cellulose derivatives, hydroxypropylmethylcellulose, hydroxypropylcellulose, hydroxymethylcellulose, hydroxyethylcellulose, methylcellulose, ethylcellulose, sodium carboxymethylcellulose, cellulose acetate, cellulose acetate phthalate, hydroxypropylmethylcellulose acetate succinate, hydroxyethylmethylcellulose, etc. may be used. Guar gum, locust bean gum, tragacantha, carrageenan, acacia gum, arabia gum, gellan gum, etc. may be used, for the gums, while for the proteins, gelatin, casein, etc. may be used. For the polyvinyl derivatives, polyvinyl alcohol, polyvinylpyrrolidone, polyvinylacetaldiethylaminoacetate, etc. may be used. As for the polymethacrylate copolymers, poly(butyl methacrylate, (2-dimethylaminoethyl)methacrylate, methylmethacrylate) copolymer, poly(methacrylic acid, methylmethacrylate) copolymer, poly(methacrylic acid, ethylacrylate) copolymer, etc. may be used, while for the polyethylene derivatives, polyethylene glycol, polyethylene oxide, etc. may be used. And, for the carboxyvinyl polymers, carbomer, etc. may be used.

When the continuous, single-phase matrix reacts with water, it becomes water swollen and gelated. When two or more water swelling type hydrophilic polymers are used, a gel layer with a smaller pore size is obtained than when a single hydrophilic polymer is used. The smaller pore size of the gel layer enables restricted release of the drug component, but, when the solubility is high and the unit dose is large as in case of metformin, it is not sufficient to maintain controlled release of wanted degree over 24 hours, by only using the use of different hydrophilic polymers in this invention.

Thus, pharmaceutically acceptable hydrophobic materials are introduced to block the diffusion of the drug through the pores.

The hydrophobic materials dispersed in the matrix block the pores of the gel layer, thereby delaying the release of the drug component, and as the pores are blocked, the matrix forms a gel phase which is more stable against erosion. Consequently, a more effective extended release can be attained using a small amount of matrix.

In the extended release system of the present invention, the use of at least two hydrophilic polymers increases the viscosity of the gel layer and reduces the pore size, and the use of the hydrophobic materials blocks the pores.

With the use of at least two water swelling type hydrophilic polymers and at least one hydrophobic material as matrix, a more ideal release profile than the conventional drugs with at least one or two water swelling type hydrophilic materials, may be obtained. Resultantly, it is possible to reduce the tablet size by using less matrix and the cost of drug production can be reduced by decreasing the usage of expensive hydrophilic polymers.

The hydrophobic materials can be classified into those with a large molecular weight and those with a small molecular weight. Examples of the pharmaceutically acceptable hydrophobic materials with a large molecular weight are polyvinyl acetate and polymethacrylate copolymers, such as poly(ethylacrylate, methyl methacrylate) copolymer, poly(ethylacrylate, methyl methacrylate, trimethylaminoethyl methacrylate) copolymer, etc. Examples of the hydrophobic materials with a small molecular weight are fatty acids and fatty acid esters like glyceryl palmitostearate, glyceryl stearate, glyceryl behenate, cetyl palmitate, glyceryl monoolate, and stearic acid, etc., fatty acid alcohols like cetostearyl alcohol, cetyl alcohol, stearyl alcohol, etc., waxes like carnauba wax, beeswax, and amorphous wax, etc., and inorganic materials like talc, precipitated calcium carbonate, calcium hydrogen phosphate, zinc oxide, titanium oxide, kaolin, bentonite, montmorillonite, and veegum, etc.

As in case of the aforementioned matrix, it may comprise the hydrophilic polymers and the hydrophobic materials with a weight proportion between 1:1 and 20:1, where the proportion between 2:1 and 15:1 is considered appropriate, and the proportion between 3:1 and 12:1 is considered ideal.

If the content of the hydrophilic polymers exceeds the above-mentioned range, the pores are not sufficiently blocked by the hydrophobic materials, resulting in increased diffusion of the drug through the pores. On the other hand, if the content of the hydrophobic materials exceeds the above-mentioned range, the gel layer of the matrix becomes too thin to include the drug.

It is recommended that the matrix is comprised in 15-75 wt %, more appropriately in 20-60 wt % and most appropriately in 30-55 wt % of the total weight of the extended release tablet. If the content of the matrix is less than 15 wt %, the drug is released too quickly, while if it exceeds 75 wt %, the drug is released too slowly and the tablet becomes too large-sized.

The metformin extended release tablet of this present invention may be prepared into the form of bare tablet or film-coated tablet. The coat layer may comprise a film-forming agent, a film-forming aid or a mixture thereof. Specifically, cellulose derivatives, sugar derivatives, polyvinyl derivatives, waxes, fats, gelatin, etc., may be used as the film-forming agent while polyethylene glycol, ethylcellulose, glycerides, titanium oxide, diethylphthalate, etc., may be used as the film-forming aid.

In case a coated tablet is prepared, it is recommended that the coat layer make up 0.5-15 wt % of the total weight of the extended release tablet.

Hereinbelow, each step of the preparation of the metformin extended release tablet in accordance with the present invention is described in detail.

In the first step, a pharmaceutical composition is prepared by mixing metformin or a pharmaceutically acceptable salt thereof, or the active ingredient, with a matrix.

Inside the matrix, the metformin and two or more hydrophilic polymers and one or more hydrophobic materials form a discontinuous matrix phase. The hydrophilic polymers swell and prolong the residence time in the gastrointestinal tract, thereby enabling sustained, controlled release of metformin included in the matrix.

The fine hydrophobic materials block the pores of the matrix layer formed by water swelling and, thus, interrupt the release of the drug through the pores.

In the second step, a diluent and an excipient are added to the mixture of the active ingredient and the matrix, and the resulting mixture is compacted to form a tablet core.

Besides the active ingredient and the matrix, the tablet core may further comprise a pharmaceutically acceptable diluent, such as starch, microcrystalline cellulose, lactose, glucose, mannitol, alginate, alkaline earth metal salts, clay, polyethylene glycol, and dicalcium phosphate, etc, within the range not affecting the purpose of the current invention. Also, starch, microcrystalline cellulose, highly dispersive silica, mannitol, lactose, polyethylene glycol, polyvinylpyrrolidone, carboxymethylcellulose, hydroxypropylmethylcellulose, hydroxypropylcellulose, natural gum, synthetic gum, copovidone, and gelatin, etc., may be used as a binder. Starches like sodium starch glycolate, cornstarch, potato starch, pre-gelatinated starch, etc., modified starches, clays like bentonite, montmorillonite, veegum, etc., celluloses like microcrystalline cellulose, hydroxypropylcellulose, carboxymethylcellulose, etc., alginates like sodium alginate, alginic acid, etc., crosslinked celluloses like croscarmellose sodium, etc., gums like guar gum, xanthan gum, etc., crosslinked polymers like crospovidone, etc., or foaming materials like sodium bicarbonate, citric acid, etc., may all be used as a disintegrator. Talc, magnesium stearate, calcium stearate, zinc stearate, lauryl sulfate, hydrogenated vegetable oil, sodium benzoate, sodium stearyl fumarate, glyceryl monostearate, polyethylene glycol 4000, etc., may all be used as a surfactant. Besides, a variety of pharmaceutically acceptable additives selected from coloring or flavoring materials may also be used.

In the following examples for this invention, light anhydrous silicic acid (Aerosil 200, Degussa, Germany), magnesium stearate, etc. were used as additive, but the scope of this invention is not limited to these additives, and the content of the scope of these additives may be determined by the one skilled in the art.

From the first and the second steps, a single-phase tablet core of the metformin extended release tablet in this invention is formed. If the fluidity of the mixture allows tabletting, a tablet may be prepared by direct compounding. Otherwise, it may be prepared through granulation processes.

In the third step, a coat layer is formed on the outer surface of the tablet core in the form of film, as required.

The metformin extended release tablet of the current invention may be administered as a bare tablet without a coat layer. The coat layer formed on the outer surface of the tablet core offers the advantage of improving the stability of the active ingredient.

The method for forming the coat layer may be selected by the one skilled in the art, for example, from fluid bed coating, pan coating, etc. Pan coating, preferably, is recommended.

The resultant metformin extended release tablet of the current invention, which is prepared by making the composition comprising metformin and a matrix into a tablet core and forming a coat layer in the form of film on the surface, enables sustained release of the drug over 24 hours at a constant rate and displays an outstanding elution characteristic to release active ingredient over 24 hours within the human body, compare to the conventional products. Thus, a constant blood level can be maintained for 24 hours with one administration a day and a desired bioequivalence can be attained.

### BRIEF DESCRIPTION OF THE DRAWINGS

FIG. 1 compares the drug release profile of the metformin extended release tablet prepared in Example 1 with that of the commercially available glucophage extended release tablet as control drug.

FIG. 2 compares the drug release profile of the metformin extended release tablet prepared in Example 1 with those of the metformin extended release tablets prepared in Comparative Examples 1 and 2.

FIG. 3 compares the drug release profile of the metformin extended release tablet prepared in Example 1 with that of the metformin extended release tablet prepared in Comparative Example 3.

FIG. 4 shows the change of the matrix structure of the gel layers of the metformin extended release tablets prepared in Example 6 and Comparative Example 4 with time [(a) Example 6: 8 hours, (b) Comparative Example 4: 8 hours, (c) Example 6: 12 hours, (d) Comparative Example 4: 12 hours, (e) Example 6: 24 hours, (f) Comparative Example 4: 24 hours].

### BEST MODE FOR CARRYING OUT THE INVENTION

This invention will be further described in detail as in the following, yet this invention is not limited by the following examples.

Practical and preferred embodiments of the present invention are illustrated as shown in the following examples. However, it will be appreciated that those skilled in the art may, in consideration of this disclosure, make modifications and improvements within the spirit and scope of the present invention.

### Example 1

### Preparation of Metformin Tablet (750 mg)

Metformin hydrochloride, polyvinylpyrrolidone, hydroxypropylmethylcellulose and glyceryl dibehenate were ground to 20 mesh and were mixed, with the composition given in Table 1. Then, light anhydrous silicic acid ground to 35 mesh was added along with magnesium stearate and was mixed. The mixture was prepared into a tablet core under a hardness condition of 15-20 kp. Subsequently, a coat film layer was formed using Opadry OY-C-7000A as the coating material with Hi-Coater (SFC-30N, Sejong Machinery, Korea) to obtain a metformin extended release tablet comprising 750 mg of metformin.

### Example 2

### Preparation of Metformin Tablet (750 mg)

Metformin hydrochloride, polyvinylpyrrolidone, carboxymethylcellulosesodium, polyvinyl alcohol and glyceryl distearate were ground to 20 mesh and mixed, with the composition given in Table 1. The mixture was prepared into a slug at 16-17 MPa, ground to 14 mesh and dried to form granules. Then, light anhydrous silicic acid ground to 35 mesh was added along with magnesium stearate and was mixed. The mixture was prepared into a tablet core under a hardness condition of 15-20 kp. Subsequently, a coat film layer was formed using Opadry OY-C-7000A as the coating material with Hi-Coater (SFC-30N, Sejong Machinery, Korea) to obtain a metformin extended release tablet comprising 750 mg of metformin.

### Example 3

### Preparation of Metformin Tablet (750 mg)

Metformin hydrochloride, polyvinylpyrrolidone, sodium carboxymethylcellulose and glyceryl dibehenate were ground to 20 mesh and were mixed, with the composition given in Table 1. Then, light anhydrous silicic acid ground to 35 mesh was added along with magnesium stearate and was mixed. A metformin extended release tablet comprising 750 mg of metformin was prepared in the same manner as in Example 1.

### Example 4

### Preparation of Metformin Tablet (750 mg)

Metformin hydrochloride, hydroxypropylmethylcellulose, polyvinyl alcohol and glyceryl dibehenate were ground to 20 mesh and were mixed, with the composition given in Table 1. A metformin extended release tablet comprising 750 mg of metformin was prepared in the same manner as in Example 2.

### Example 5

### Preparation of Metformin Tablet (750 mg)

Metformin hydrochloride, polyvinylpyrrolidone, sodium carboxymethylcellulose, sodium alginate and glyceryl dibehenate were ground to 20 mesh and were mixed, with the composition given in Table 1. Then, light anhydrous silicic acid ground to 35 mesh was added along with magnesium stearate and was mixed. A metformin extended release tablet comprising 750 mg of metformin was prepared in the same manner as in Example 1.

### Example 6

### Preparation of Metformin Tablet (750 mg)

Metformin hydrochloride, hydroxypropylmethylcellulose and Kollidon SR were ground to 20 mesh and were mixed, with the composition given in Table 1. Then, light anhydrous silicic acid ground to 35 mesh was added along with magnesium stearate and was mixed. A metformin extended release tablet comprising 750 mg of metformin was prepared in the same manner as in Example 1.

### Comparative Example 1

### Preparation of Metformin Tablet (750 mg)

Metformin hydrochloride and polyvinylpyrrolidone were ground to 20 mesh and were mixed, with the composition given in Table 1. Then, light anhydrous silicic acid ground to 35 mesh was added along with magnesium stearate and was mixed. A metformin extended release tablet comprising 750 mg of metformin was prepared in the same manner as in Example 1.

### Comparative Example 2

### Preparation of Metformin Tablet (750 mg)

Metformin hydrochloride, polyvinylpyrrolidone and hydroxypropylmethylcellulose were ground to 20 mesh and were mixed, with the composition given in Table 1. Then, light anhydrous silicic acid ground to 35 mesh was added along with magnesium stearate and was mixed. A metformin extended release tablet comprising 750 mg of metformin was prepared in the same manner as in Example 1.

### Comparative Example 3

### Preparation of Metformin Tablet (750 mg)

Metformin hydrochloride and hydroxypropylmethylcellulose were ground to 20 mesh and were mixed, with the composition given in Table 1. Then, light anhydrous silicic acid ground to 35 mesh was added along with magnesium stearate and was mixed. A metformin extended release tablet comprising 750 mg of metformin was prepared in the same manner as in Example 1.

### Comparative Example 4

### Preparation of Metformin Tablet (750 mg)

Metformin hydrochloride and hydroxypropylmethylcellulose were ground to 20 mesh and were mixed, with the composition given in Table 1. Then, light anhydrous silicic acid ground to 35 mesh was added along with magnesium stearate and was mixed. A metformin extended release tablet comprising 750 mg of metformin was prepared in the same manner as in Example 1.

TABLE 1

| | Composition (mg/tablet) | | | | | | | | | |
|---|---|---|---|---|---|---|---|---|---|---|
| | Example # | | | | | | Comparative Example # | | | |
| Constituents | 1 | 2 | 3 | 4 | 5 | 6 | 1 | 2 | 3 | 4 |
| Metformin hydrochloride | 750 | 750 | 750 | 750 | 750 | 750 | 750 | 750 | 750 | 750 |
| Polyvinylpyrrolidone | 120 | 120 | 240 | — | 120 | — | 390 | 150 | — | — |
| Hydroxypropylmethylcellulose¹⁾ | 240 | — | — | 240 | — | 240 | | 240 | 500 | 390 |
| Sodium carboxymethylcellulose²⁾ | — | 120 | 120 | — | 120 | — | — | — | — | — |
| Polyvinyl alcohol | — | 120 | — | 120 | — | — | — | — | — | — |
| Sodium alginate | — | — | — | — | 120 | — | — | — | — | — |
| Kollidon SR³⁾ | — | — | — | — | — | 150 | — | — | — | — |
| Glyceryl dibehenate⁴⁾ | 30 | — | 30 | 30 | 30 | — | — | — | — | — |
| Glyceryl distearate⁵⁾ | — | 30 | — | — | — | — | — | — | — | — |
| Aerosil 200 | 7.5 | 7.5 | 7.5 | 7.5 | 7.5 | 7.5 | 7.5 | 7.5 | 7.5 | 7.5 |
| Magnesium stearate | 7.5 | 7.5 | 7.5 | 7.5 | 7.5 | 7.5 | 7.5 | 7.5 | 7.5 | 7.5 |
| Opadry OY-C-7000A⁶⁾ | 60 | 60 | 60 | 60 | 60 | 60 | 60 | 60 | 60 | 60 |
| Total | 1215 | 1215 | 1215 | 1215 | 1215 | 1215 | 1215 | 1215 | 1325 | 1215 |

¹⁾Dow Chemical, U.S.
²⁾Bo Lak, Korea.
³⁾BASF, Germany; 2:8 mixture of polyvinylpyrrolidone (hydrophilic polymer) and polyvinylacetate (hydrophobic material).
⁴⁾Gatefosse, France.
⁵⁾Gatefosse, France.
⁶⁾Colorcon, U.S.

### Testing Example 1

### Comparative Dissolution Profile Test

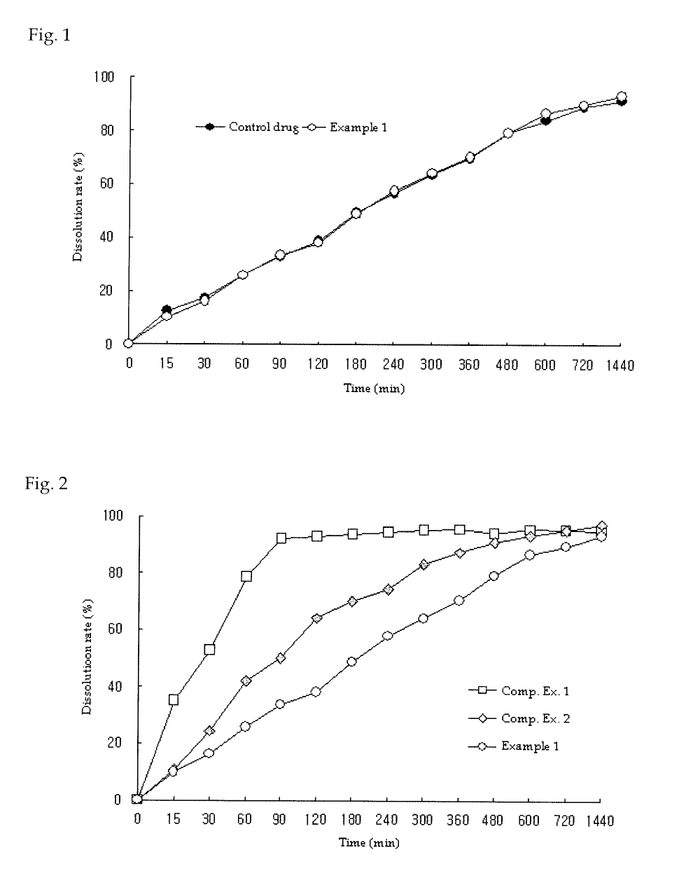

Dissolution properties were tested for the metformin extended release tablet of the present invention prepared in Example 1 and commercially available Glucophage XL (BMS, U.S.) as control drug according to the paddle dissolution profile test method of Korean Pharmacopoeia. The result is shown in FIG. 1.

As seen in FIG. 1, the metformin extended release tablet of the present invention showed a dissolution profile comparable to that of the control drug. While the control drug (Korean Patent Application No. 2000-7010280) controls drug release with a two-phase matrix, the tablet of the present invention maintains systematic drug release with a single-phase matrix, which simplifies the manufacturing method and process and reduces manufacturing time. And, the extended release tablet of the present invention has a uniform dissolution profile.

### Testing Example 2

### Comparative Dissolution Profile Test

Dissolution properties were tested for the metformin extended release tablets prepared in Example 1, Comparative Example 1 and Comparative Example 2 according to the paddle dissolution profile test method of Korean Pharmacopoeia. The result is shown in FIG. 2.

The total weight of the matrix used in Example 1, Comparative Example 1 and Comparative Example 2 was the same.

As seen in FIG. 2, the metformin extended release tablet of Example 1, in which the matrix comprised polyvinylpyrrolidone and hydroxypropylmethylcellulose as hydrophilic polymer and glyceryl dibehenate as hydrophobic material, showed significantly better extended release than the tablet of Comparative Example 1, in which the matrix comprised polyvinylpyrrolidone only, or the tablet of Comparative Example 2, in which the matrix comprised polyvinylpyrrolidone and hydroxypropylmethylcellulose. While the tablet of Comparative Example 1 showed better extended release than that of Comparative Example 2, it was worse than that of Example 1.

It can be inferred that as the matrix comprised polyvinylpyrrolidone and hydroxypropylmethylcellulose as hydrophilic polymer, it swells in the gastric juice by water swelling, thereby resulting in fine pores, which are blocked by glyceryl dibehenate, or the hydrophobic material, and the diffusion of the drug is further delayed.

### Testing Example 3

### Comparative Physical Property Test

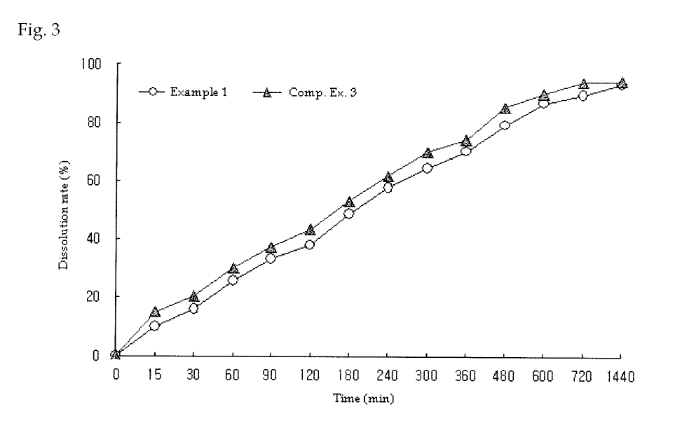

In Comparative Example 3, excessive matrix was added and a dissolution profile similar to that of Example 1 was obtained (see FIG. 3), compared with other Comparative Examples. Total volume and weight of the tablet and the weight of matrix were compared in Table 2 below.

TABLE 2

| | Matrix | Table | Tablet volume |
|---|---|---|---|
| | weight (mg) | weight (mg) | (mL/tablet) |
| Example 1 | 390 | 1215 | 0.98 |
| Comparative Example 3 | 500 | 1325 | 1.275 |

As seen in Table 2, less matrix was used in Example 1 than in Comparative Example 3 and total weight and volume of the tablet were much smaller, too.

Since extended release can be maintained with less matrix, it seems to be the perfect matrix system for metformin which requires a large unit dose. The smaller tablet volume improves patients' convenience in medication and the use of less matrix offers an economical advantage.

### Testing Example 4

### Microscopic Observation of Matrix Structure

The matrix structure of the gel layers of the metformin extended release tablets prepared in Example 6 and Comparative Example 4 was observed with an optical microscope (Olympus 1×70, ×100). The samples were swelled in water for 8, 12 and 24 hours, lyophilized and observed with the optical microscope.

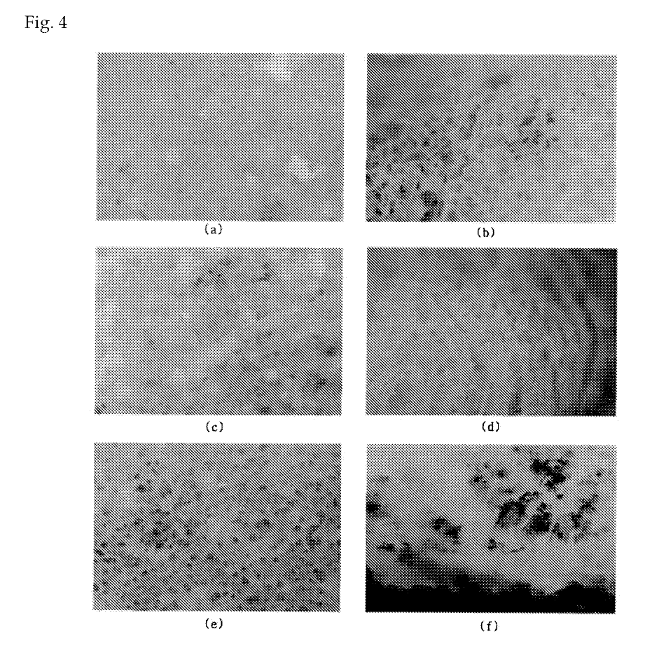

As the released drug is diffused into the swollen, gelated matrix through the pores of the gel layer, the gel layer is eroded and the pores become larger, resulting in the reduction of viscosity and elasticity of the gel layer. As seen in FIG. 4, the pore size increased with time and the matrix structure collapsed after 24 hours.

As seen in the figure, Example 6 had a smaller pore size than Comparative Example 4 and showed a tighter matrix structure as the hydrophobic material (seen white in the figure) blocked the pores. And, the small pore size was maintained after 6, 12 and 24 hours. While the matrix almost collapsed after 24 hours in Comparative Example 4, that of Example 6 maintained its structure, which confirms its stability against erosion.

The pore size was smaller when hydroxypropylmethylcellulose and Kollidon SR were used than when a single hydrophilic polymer was used. It may be due to the blocking of pores and the interruption of diffusion of metformin by the polyvinylacetate, or the hydrophobic material.

### INDUSTRIAL APPLICABILITY

As apparent from the above description, the present invention offers an ideal administration form for metformin, a drug effective in treating non-insulin dependent diabetes mellitus, which is highly soluble in water, has a narrow absorption window and requires a large unit dose.

The metformin extended release tablet of the present invention shows superior dissolution properties and is capable of releasing the drug component slowly at a constant rate while using less matrix than conventional extended release tablets. Thus, the blood level can be maintained constant over 24 hours with a single administration a day. In addition, the smaller tablet volume improves patients' convenience in administration and the use of less matrix offers an economical advantage.

Those skilled in the art will appreciate that the concepts and specific embodiments disclosed in the foregoing description may be readily utilized as a basis for modifying or designing other embodiments for carrying out the purposes of the present invention. Those skilled in the art will also appreciate that such equivalent embodiments do not depart from the spirit and scope of the present invention as set forth in the appended claims.

1. A metformin extended release tablet comprising metformin as active ingredient and a pharmaceutically acceptable hydrophilic polymer and a hydrophobic material as matrix.

2. The metformin extended release tablet as set forth in claim 1, wherein the metformin is in the form of an acid adduct.

3. The metformin extended release tablet as set forth in claim 2, wherein the metformin is metformin hydrochloride.

4. The metformin extended release tablet as set forth in claim 1, wherein the metformin constitutes 25-75 wt % of the total weight of the extended release tablet.

5. The metformin extended release tablet as set forth in claim 1, wherein the hydrophilic polymer is at least two selected from saccharides, gums, proteins, polyvinyl derivatives and acrylate derivatives.

6. The metformin extended release tablet as set forth in claim 5, wherein the hydrophilic polymer is at least two selected from saccharides including dextrin, polydextrin, dextran, pectin, a pectin derivative, alginate, polygalacturonic acid, xylan, arabinoxylan, arabinogalactan, starch, hydroxypropyl starch, amylose, amylopectin, etc., cellulose derivatives including hydroxypropylmethylcellulose, hydroxypropylcellulose, hydroxymethylcellulose, hydroxyethylcellulose, methylcellulose, ethylcellulose, sodium carboxymethylcellulose, cellulose acetate, cellulose acetate phthalate, hydroxypropylmethylcellulose acetate succinate, hydroxyethylmethylcellulose, etc., gums including guar gum, locust bean gum, tragacantha, carrageenan, acacia gum, arabia gum, gellan gum, etc., proteins like gelatin, casein, etc., polyvinyl derivatives including polyvinyl alcohol, polyvinylpyrrolidone, polyvinylacetal diethylaminoacetate, etc., polymethacrylate copolymers including poly(butyl methacrylate, (2-dimethylaminoethyl)methacrylate, methylmethacrylate) copolymer, poly(methacrylic acid, methylmethacrylate) copolymer, poly(methacrylic acid, ethylacrylate) copolymer, etc., polyethylene derivatives including polyethylene glycol, polyethylene oxide, etc. and carboxyvinyl polymers including carbomer, etc.

7. The metformin extended release tablet as set forth in claim 1, wherein the hydrophobic material is at least one selected from a pharmaceutically acceptable hydrophobic material with a large molecular weight like polyvinyl acetate, polymethacrylate copolymer including poly(ethylacrylate, methyl methacrylate) copolymer, poly(ethylacrylate, methyl methacrylate, trimethylaminoethylmethacrylate) copolymer, etc., a hydrophobic material with a small molecular weight like fatty acid or fatty acid ester, including glyceryl palmitostearate, glyceryl stearate, glyceryl behenate, cetyl palmitate, glyceryl monoolate, stearic acid, etc., fatty acid alcohol including cetostearyl alcohol, cetyl alcohol, stearyl alcohol, etc., wax including carnauba wax, beeswax, amorphous wax, etc., and inorganic material including talc, precipitated calcium carbonate, calcium hydrogen phosphate, zinc oxide, titanium oxide, kaolin, bentonite, montmorillonite, veegum, etc.

8. The metformin extended release tablet as set forth in claim 1, wherein the hydrophilic polymer and the hydrophobic material are comprised in 1:1-20:1 by weight.

9. The metformin extended release tablet as set forth in claim 1, wherein the matrix constitutes 15-75 wt % of the total weight of the extended release tablet.

10. The metformin extended release tablet as set forth in claim 1, which is a bare tablet or a coated tablet having a coat layer in the form of film.

11. The metformin extended release tablet as set forth in claim 10, wherein the coat layer comprises a film-forming agent, a film-forming aid or a mixture thereof.

12. The metformin extended release tablet as set forth in claim 11, wherein the coat layer comprises at least one selected from a cellulose derivative, a sugar derivative, a polyvinyl derivative, a wax, a fat, gelatin, polyethylene glycol, ethylcellulose, a glyceride, titanium oxide and diethylphthalate.

13. The metformin extended release tablet as set forth in claim 10, wherein the coat layer constitutes 0.5-15 wt % of the total weight of the extended release tablet.
